(19) United States
(12) Patent Application Publication
(10) Pub. No.: US 2005/0125013 A1
(43) Pub. Date: Jun. 9, 2005

(54) Safety surgical forceps

(76) Inventor: Alan Kessler, Los Angeles, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/987,939

(22) Filed: Nov. 12, 2004

Related U.S. Application Data
(60) Provisional application No. 60/519,959, filed on Nov. 14, 2003.

(51) Int. Cl. A61B 17/44; A61B 17/04

(52) U.S. Cl. 606/148; 606/205

(57) ABSTRACT

Surgical forceps, which have first and second elongate arms, are provided with gripping pads near the distal ends of the arms. The gripping pads are resilient so as to conform to the shape of an object being gripped between the arms. The surgical forceps is preferably provided with a locking mechanism for maintaining the forceps in the closed position in the absence of a gripping force. The surgical forceps are particularly well suited for gripping a suturing needle in a secure and predictable manner. The gripping pads are preferably sized for enclosing the distal pointed tip of the suturing needle. As a result, the forceps may be locked in the closed position with the needle tip fully enclosed for safe disposal.

### RELATED APPLICATIONS

The present application claims priority under 35 U.S.C. §119(e) to U.S. Provisional Application No. 60/519,959, filed Nov. 14, 2003, which is hereby incorporated by reference in its entirety.

### BACKGROUND OF THE INVENTION

1. Field of the Invention

The invention relates generally to surgical instruments and, more particularly, to surgical forceps and related instruments used in conjunction with needles for applying sutures.

2. Description of the Related Art

Surgical forceps typically include a pair of elongate opposing arms that are joined along the proximal ends. The distal ends of the opposing arms provide jaws configured for gripping a variety of objects, such as bodily tissue, organs, blood vessels and medical instruments. The jaws may be formed with teeth for increasing friction and firmly gripping the object.

In one common application, surgical forceps are used when suturing closed a wound. In this application, the clinician pushes the pointed tip of a suturing needle into and through the tissue using a surgical instrument (e.g., a needle holder or driver). As the pointed tip of the needle emerges from the tissue, the clinician grasps the needle tip with the forceps for pulling the needle completely through and out of the tissue. Clinicians prefer to use forceps for gripping and pulling a suturing needle for a variety of reasons, such as, for example, to improve the gripping force, to avoid contamination and to avoid being stuck.

Although widely used, existing surgical forceps suffer from a variety of shortcomings, particularly when used in conjunction with suturing needles. One primary shortcoming arises because the shape of the needle may not be compatible with the jaws of the forceps. Suturing needles are typically formed with a body that has a curved, bent, or hooked shape to facilitate passing the needle into and out of the tissue. In addition, suturing needles are often formed with a non-circular cross-section, such as, for example, a triangular, square or tapered cross-section. On the other hand, the jaws of the forceps are typically provided with substantially flat opposing surfaces. As a result, it has been found in practice that a suturing needle often has a tendency roll or rotate along its longitudinal axis when a compressive force is applied (i.e., when squeezed between the arms of the forceps). When a curved needle inadvertently rolls onto its side during the application of sutures, the distal pointed tip of the needle may become flush with the surface of the tissue. In an attempt to reposition a rolled needle, the clinician may attempt to grasp the needle with the forceps at a different angle, which requires significant dexterity which can cause damage to the tissue. Alternatively, the clinician may attempt to grab or flick the needle with his or her fingers to reposition the needle such that the tip is not pressed against the tissue. However, adjusting the position of the needle with one's fingers is difficult and dangerous because there is a substantial likelihood of an accidental needle stick. Accordingly, when a needle rolls, it may become difficult or impossible for the clinician to continue pulling the needle without great difficulty.

Accidental needle sticks also can occur while pulling the needle through the tissue with the forceps. In the process of pulling the needle with the forceps, the pointed needle tip may accidentally stick the clinician, the patient or an assistant.

Needle sticks may also occur when the needle is transferred from the forceps to a waste container for disposal. During the transfer of the needle to the waste container, a clinician or an aid may grab the needle with his or her fingers, at which time a needle stick can occur. Furthermore, after the needle is transferred to the waste container, the contaminated needle stills present a serious hazard to others when disposing and processing the waste.

Using forceps to manipulate a suturing needle additionally can damage the delicate gripping surfaces along the jaws of the forceps, thereby rendering the forceps useless for subsequent procedures. Furthermore, it has been found that the gripping surfaces of the forceps can bend or damage the tip of the suturing needle. This is particularly disadvantageous because a bent or damaged needle tip may damage the tissue as the needle is used on successive suture passes. Moreover, if the forceps are used to grasp the suture, the hard gripping surfaces of the opposing arms may damage or cut the suture, thereby requiring the sutures to be removed and the entire process repeated.

### SUMMARY OF THE INVENTION

Embodiments of the present invention relate to surgical instruments or similar tools used for gripping and holding articles and devices, such as, for example, suturing needles. Various embodiments of the present invention can address some or all of the noted shortcomings associated with existing forceps. For example, and in accordance with the present invention, a pair of surgical forceps is provided with an improved configuration and structure that is better suited for gripping a suturing needle without damaging the forceps or the needle. The forceps is preferably adapted to reduce the likelihood of needle sticks and is configured to grasp a needle in a secure manner such that the needle will not roll or rotate in an undesirable manner when a gripping force is applied. The forceps are also preferably configurable for grasping a needle such that the pointed needle tip is partially or fully enclosed within the gripping portion of the forceps as the clinician or other healthcare provider pulls the needle and suture through the tissue. Preferred embodiments of the forceps are reliable, are convenient to use, and significantly improve the efficacy and safety of a wide variety of procedures.

In accordance with one aspect of the present invention, a forceps has first and second elongate arms that are joined together, preferably along a proximal end portion. First and second gripping surfaces are disposed on the inner faces of the first and second arms along the distal end portions. The first and second gripping surfaces are disposed in an opposing relationship for holding an object therebetween when an external gripping force is applied to the forceps. One or both of the gripping surfaces preferably includes a gripping pad made of a resilient material that conforms to the shape of the object being held. Due to the malleable (i.e., easily deformable) nature of the resilient material, the pad provides a substantially even gripping force along the surface of the object.

In a preferred mode, each arm includes an expanded area near its distal end at which the corresponding gripping surface is disposed. However, the forceps can have one or more gripping surfaces along the arm(s) of forceps, with or without expansion of the grasping surface and may be on one or both opposing surfaces. Additionally, the opposing gripping surfaces on the inner sides of the arms, which can include the gripping pad(s), can be of different shapes and sizes.

A biasing mechanism can be provided in some applications for biasing the distal end portions of the arms apart in the absence of a gripping force. Additionally, in some applications, the resilient material of the gripping pad(s) can be textured (e.g., can be formed to have dimples, ridges, protuberances, and like formations). In a preferred form, the forceps are configured to grasp a suturing needle.

Surgical forceps including the forgoing features are particularly advantageous for use in conjunction with a suturing needle. The forceps allow the clinician to grip and hold the needle in a secure and predictable manner. In one important feature, the resilient gripping pad(s) prevent undesirable rotational movement of the needle (or other object) with respect to the forceps. The resilient gripping pad(s) are preferably sufficiently soft to a clinician to use the forceps to grasp the suture without fraying or otherwise damaging the suture.

In accordance with another aspect of the present invention, the surgical forceps have first and second elongate arms that are joined along a proximal end portion. First and second gripping pads are preferably provided on the inner faces of the first and second arms along the distal end portions. The gripping pads are formed with a relatively large size such that at least the tip of a suturing needle may be enclosed between the pads when the needle is gripped with the forceps. In a more preferred form, the gripping pads of the surgical forceps include resilient material that deforms about at least a portion of the suturing needle when the forceps grasp the needle tip.

In accordance with another aspect of the invention, the forceps may be provided with a first gripping pad made of a resilient material and a second gripping surface made of a more rigid (e.g., metallic) material such that the needle is pressed between relatively hard and soft surfaces. The resilient material is configured to at least partially conform to the shape of the needle to prevent the needle from rolling or otherwise moving while being gripped with the forceps.

Another aspect of the invention involves a surgical forceps in which the tissue grasping end of the forceps is structurally retained in shape but a resilient coating (e.g., an elastomeric coating) covers at least one or both of the opposing inner surfaces of the forceps' distal ends. These and other aspects of surgical forceps as generally described herein can significantly reduce the likelihood of accidental needle sticks and provide a major advancement in the field of surgical forceps. In addition, when used with forceps that include a gripping structure for tissue manipulation (e.g., teeth), the use of resilient gripping pads allows the clinician to grasp a needle without damaging the gripping structure.

A further aspect of the invention involves a surgical instrument (e.g., forceps) that is provided with a locking mechanism for holding opposing pads of the instrument together in the absence of a gripping force. When used with surgical forceps, the locking mechanism may take the form of a clasp or a sliding ring disposed along the proximal end portions of first and second arms. The sliding ring preferably surrounds the arms and slides forward (i.e., toward the distal ends) for holding the gripping surfaces together. The sliding ring may be provided with a retaining mechanism to hold the sliding ring in a set forward position. Accordingly, the forceps may be disposed with the arms locked and with a contaminated needle held safely and securely between the opposing surfaces.

An additional aspect of the present invention involves a pair of forceps for gripping and retaining a object (e.g., a surgical needle). The forceps comprise first and second arms that proximal and distal end portions. The arms are joined together at least near the proximal end portions, and preferably at the proximal ends thereof. First and second gripping surfaces are disposed along the distal end portions of the first and second arms, respectively, either at or proximal of the distal ends. At least the first gripping surface includes a first resilient gripping pad. The gripping surfaces are disposed in an opposing relationship for gripping an object therebetween when a gripping force is applied to the forceps.

In accordance with another aspect of the present invention, there is provided a pair of forceps for gripping and retaining a surgical needle. The forceps comprise first and second arms having proximal and distal end portions. The arms are joined together at least near the proximal end portions, and preferably at the proximal end thereof. First and second gripping surfaces are disposed on the first and second arms in an opposing relationship for holding an object therebetween when a gripping force is applied to the forceps. A locking mechanism is also provided for selectively maintaining the distal end portions of the first and second arms in close proximity for gripping the object when the gripping force is removed.

Another aspect of the present invention involves forceps for gripping and retaining a surgical needle. The forceps comprise first and second arms having proximal and distal end portions and being joined together at least near the proximal end portions. First and second gripping surfaces are disposed at first and second enlarged regions located along the distal end portions of the first and second arms, respectively. The gripping surfaces are disposed in an opposing relationship for gripping an object therebetween when a gripping force is applied to the forceps.

An additional aspect of the present invention involves forceps comprising a first arm that has a proximal end and a distal end. The first arm also has first and second faces disposed on opposite sides of the distal end. A second arm similarly has a proximal end and a distal end with first and second faces disposed on opposite sides of the distal end. The proximal ends of the first and second arms are rotatably coupled such that the forceps has a first configuration, in which an object can be gripped between the first faces of the first and second arms, and a second configuration in which an object can be gripped between the second faces of the first and second arms. In a preferred mode, at least the first faces each comprise a resilient pad.

A further aspect of the invention involves a method of closing a wound in which surgical forceps are provided that have first and second elongate arms; the arms have first and second resilient pads disposed along inner faces in an opposing relationship. A suturing needle is inserted through a patient's tissue on opposite sides of the wound such that a distal tip of the suturing needle emerges from the tissue with the suturing needle pulling suture through the tissue. The distal tip of the suturing needle is gripped between the first and second resilient gripping pads and the needle is drawn through the tissue. The surgical forceps are locked in a closed position such that at least the distal tip of the needle is fully enclosed and protected between the resilient pads. The needle and forceps are disposed together.

### BRIEF DESCRIPTION OF THE DRAWINGS

These and other features, aspects and advantages are described with reference to drawings of several preferred embodiments of the present invention, which are intended to illustrate, but not to limit, the present invention. The drawings include 27 figures.

FIG. 1 is a perspective view of a preferred embodiment of a surgical forceps provided with gripping pads and a locking mechanism;

FIG. 2 is a top view of the surgical forceps illustrated in FIG. 1, with a textured surface omitted from the drawings;

FIG. 3 is a side view of the surgical forceps of FIG. 1 shown in an open position;

FIG. 4 is a side view of the surgical forceps of FIG. 1 shown in a closed and locked position;

FIG. 5 is an enlarged plan view illustrating an inner distal portion of a first arm of the forceps of FIG. 1;

FIG. 6 is an enlarged plan view illustrating the distal portion of a second arm of the forceps of FIG. 1;

FIG. 7 is a cross-sectional view of the second arm of FIG. 6;

FIG. 8 is an enlarged side view illustrating the distal end portions of the forceps of FIG. 1 in the closed position;

FIG. 8A is an enlarged side view illustrating the distal end portion of an another embodiment, wherein a gripping pad is provided on only the second arm;

FIG. 9 is a plan view illustrating the top side of a preferred locking mechanism configured for use with the forceps of FIG. 1;

FIG. 10 is a side cross-sectional view of the locking mechanism of FIG. 9;

FIG. 11 is a bottom view of the locking mechanism of FIG. 9;

FIG. 12 is a perspective view illustrating the surgical forceps of FIG. 1 in the open position with a suturing needle;

FIG. 12A is a perspective view illustrating the surgical forceps of FIG. 1 in the closed position such that the tip of the needle is enclosed between the gripping pads;

FIG. 13 is a perspective view of another preferred embodiment of a surgical forceps provided with gripping pads and having an alignment pin and a clasp for locking the forceps in the closed position;

FIG. 14 is a side elevational view illustrating the surgical forceps of FIG. 13 in the closed and locked position;

FIG. 15 is an plan view illustrating the inner face of the first arm of the surgical forceps of FIG. 13;

FIG. 16 is a cross-sectional view of the distal end portion of the forceps of FIG. 15;

FIG. 17 is an enlarged view illustrating the inner face of the first arm of the surgical forceps of FIG. 14;

FIG. 18 is a cross-sectional view illustrating an additional type of gripping pads for use with the forceps of FIG. 14;

FIG. 19 is an enlarged view illustrating the inner face of the first arm of the surgical forceps of FIG. 18;

FIG. 20 is an enlarged view illustrating an alignment pin configured for use with the surgical forceps of FIG. 14;

FIG. 21 is a side view illustrating another embodiment of a surgical forceps wherein the arms are reversible and each arm is provided with teeth on a first side and gripping pads on a second side;

FIG. 22 is a plan view illustrating the surgical forceps of FIG. 21 when reversing the orientation of the arms;

FIG. 23 is a plan view illustrating the surgical forceps of FIG. 21 in a reversed position such that the gripping pads are located along the inner faces of the arms;

FIG. 24A through 24C are side cross-sectional views illustrating various optional tip configurations for use with the surgical forceps of the present invention and illustrating another embodiment of the gripping pads;

FIG. 25A is a perspective view illustrating a preferred biasing mechanism for use with the surgical forceps of FIG. 14;

FIG. 25B is a cross-sectional view illustrating the biasing mechanism of FIG. 25A;

FIG. 26 is a side cross-sectional view illustrating the distal end portion of a further embodiment of a surgical forceps wherein the gripping pads are formed with a V-shape; and

FIG. 27 is an enlarged perspective view of the first opposing pad of FIG. 26.

### DETAILED DESCRIPTION OF THE PREFERRED EMBODIMENT

Embodiments of the present invention provide surgical forceps that improves a clinician's ability to grip a wide variety of objects in a safe and secure manner. The forceps also improve the clinician's ability to manipulate objects without damaging the object or the forceps. Preferred embodiments are particularly well-suited for gripping and manipulating a suturing needle during the application of sutures. However, it should be appreciated that embodiments of the described forceps and/or features thereof may be used in a wide variety of applications.

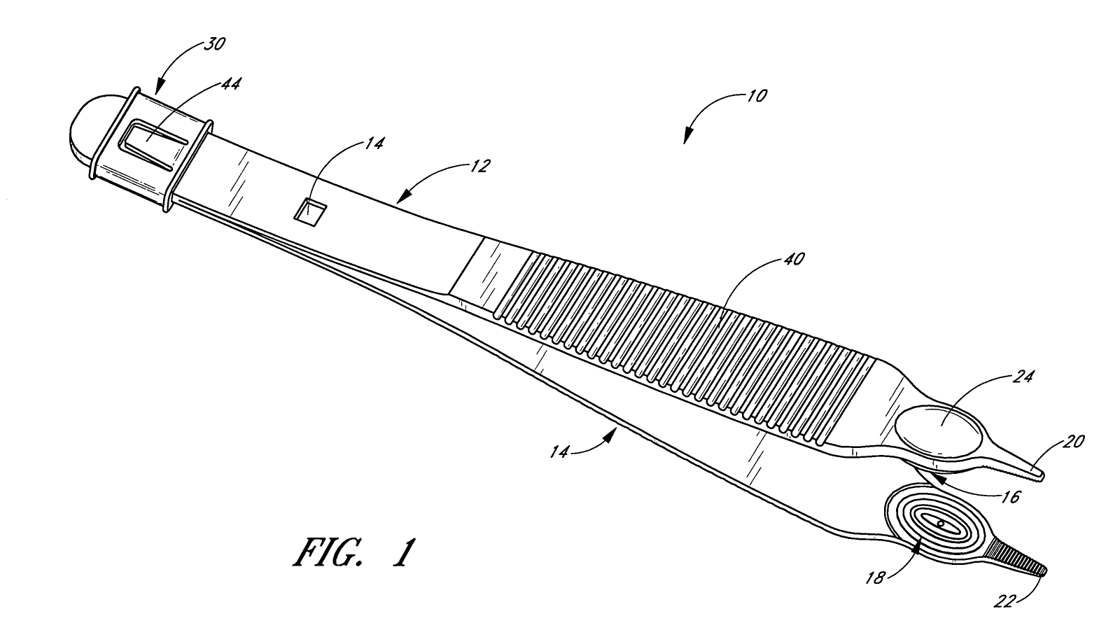

With reference initially to FIG. 1, for purposes of illustration, one embodiment of a surgical forceps 10 generally includes a first elongate arm 12 and a second elongate arm 14 joined along proximal ends thereof. The first and second arms 12, 14 are preferably joined such that the arms are biased in the open position in the absence of an external gripping force by the clinician. In the illustrated embodiment, the arms are rigidly joined together. However, the arms can be interconnected in a variety of other ways. For example, in one variation, the first and second arms can be rotatably coupled in a manner similar to a hemostatic forceps.

The first and second arms 12, 14 are preferably substantially flat elongate members having opposing distal end portions configured for gripping an object. First and second gripping surfaces are provided along the inner faces of the first and second arms 12, 14, respectively. In one preferred feature, the first and second gripping surfaces comprise first and second gripping pads 16, 18. The first and second gripping pads 16, 18 are disposed in an opposing relationship for holding an object, such as a suturing needle. The gripping pads 16, 18 are preferably made of a resilient material such that the gripping pads deform when a gripping force is applied to the object. As a result of the deformation, the gripping pads at least partially conform to the shape of the object held by the forceps. The resiliency and arrangement of the gripping pads provides a variety of advantages that will be discussed in more detail below.

In the illustrated embodiment, the first and second arms 12, 14 further comprise opposing tips 20, 22 configured for gripping objects, such as tissue, in a very secure manner. In various preferred embodiments, the opposing tips may be straight, curved, serrated, toothed or may have any other configuration well-suited for gripping. The opposing tips 20, 22 shown in FIG. 1 are provided with serrated teeth along the inner faces. In one application, the opposing tips may be used to grip and pull tissue while a needle is passed through the tissue for applying sutures. While the gripping pads 16, 18 in this embodiment are disposed proximal of the tips 20, 22, the gripping pads 16, 18 can be disposed at the distal tips 20, 22 in other applications.

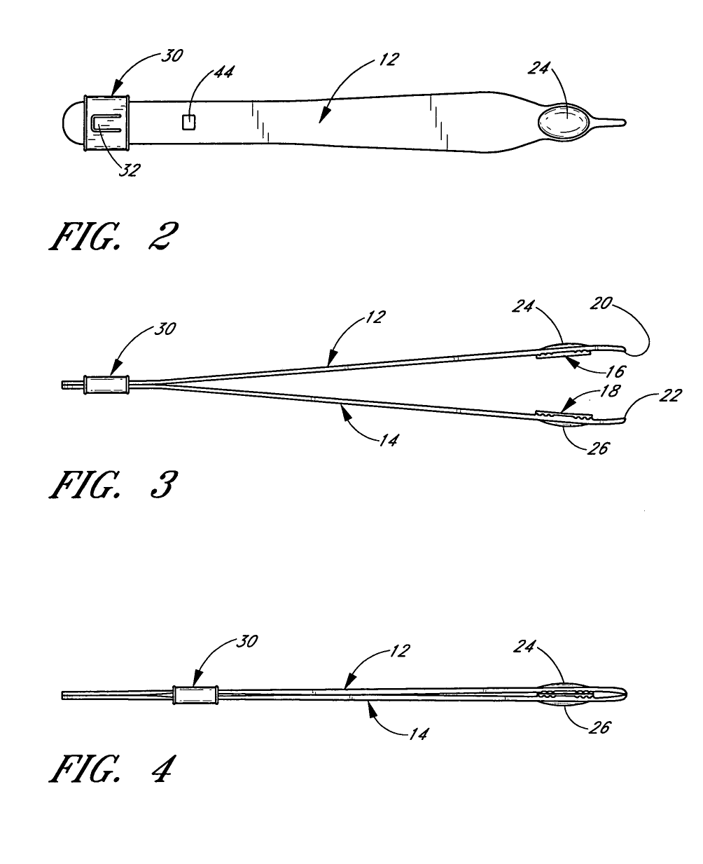

The outer surface of each of the arms can be smooth or can be provided with a textured surface 40 for providing an improved tactile sense and enhancing the clinician's ability to grip and hold the forceps during use. The textured surface(s) may take the form of ridges or serrations provided along a central section of the arms. The textured surface(s) may be formed into the surfaces of the arms by molding or machining. Alternatively, the textured surface(s) may be provided as a separate material disposed along the outer surfaces of the arms. The textured surface(s) are particularly valuable for preventing slippage when the physician is wearing surgical gloves. In addition, the outer surface of each can may be provided with an outer pad 24 along the distal end portion. The outer pad(s) provide improved comfort during use and further enhance friction for improved handling. The outer pad(s) 24 are particularly well located for squeezing the forceps with a thumb and forefinger. The first outer pad 24 and the second outer pad 26 can each be seen in the side view of the forceps 10 shown in FIG. 3.

With continued reference to FIG. 1, a locking mechanism 30 preferably is provided along the proximal end portions of the first and second arms 12, 14. The locking mechanism preferably surrounds the first and second arms and is configured for longitudinally movement. The locking mechanism 30 preferably includes a retaining mechanism 32 that is receivable within a slot or opening 44 formed in the first arm 12. The locking mechanism has a proximal position wherein the forceps 10 are in an open (i.e., operable) condition. The locking mechanism also has a distal position wherein the forceps 10 are locked in the closed position. With reference now to FIGS. 2 and 3, top and side views of the surgical forceps 10 are provided. In each of these views, the locking mechanism is in the proximal position and the first and second arms 12, 14 are in the open position.

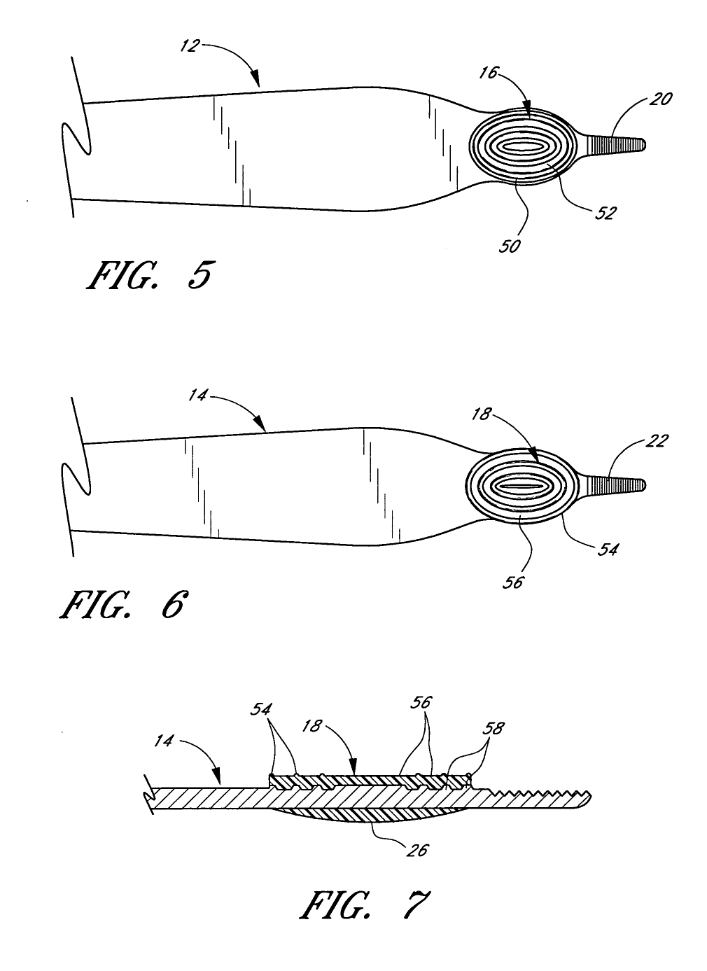

With reference now to FIG. 4, the surgical forceps is shown with the locking mechanism 30 slid forward such that the forceps are locked in the closed position. When in the closed position, it can be seen that the first and second arms are held together such that the gripping pad 16 of the first arm 12 is preferably maintained in firm contact with the gripping pad 18 of the second arm 14. When slid forward, the retaining mechanism 32 of the locking mechanism 30 is captured and held within the opening 44 formed in the first arm. As a result, the locking mechanism 30 is prevented from sliding back in a proximal direction. Accordingly, by using the locking mechanism 30, the surgical forceps may be quickly and easily locked in the closed position by the clinician. As will be discussed in more detail below, the locking mechanism feature is particularly advantageous for use with suturing needles wherein the distal tip of the needle can be safely enclosed and retained within the forceps for safe disposal.

With reference now to FIG. 5, an enlarged view of the inner face of the first arm 12 is shown. It can be seen that the first gripping pad 16 is disposed near the distal end along an enlarged (i.e., wide) portion of the first arm 12. In one embodiment, the gripping pad 16 may be an insert that is glued or molded on to the arm. In the region distal to the gripping pad 16, the first arm narrows to form the tip portion 20. It can be seen that the first gripping pad 16 may be formed with a plurality of concentric oval ridges 50. Corresponding grooves 52 are provided between the ridges 50. In one preferred embodiment, the largest oval ridge 50 has a length of about 0.4 inches and a width of about 0.3 inches.

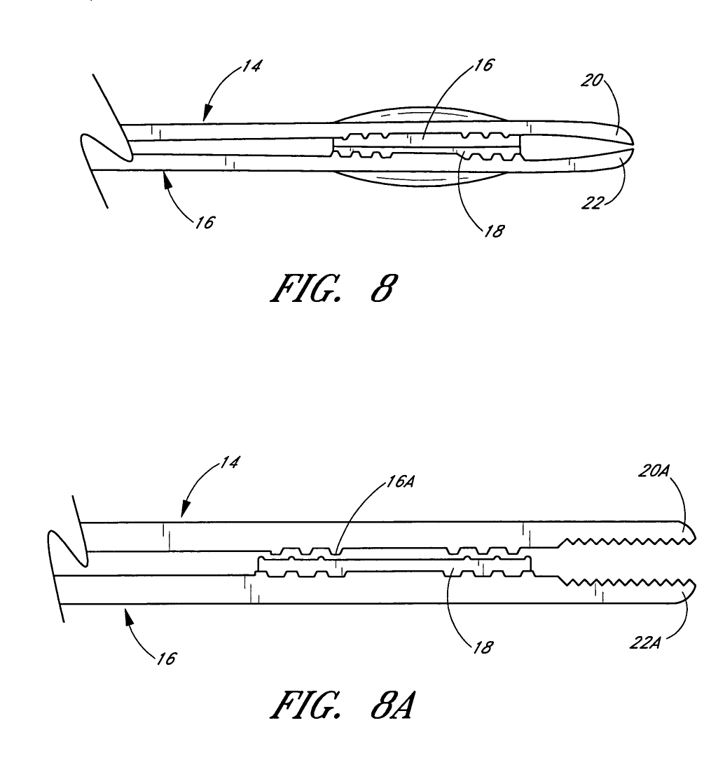

With reference now to FIG. 6, an enlarged view of the inner face of the second arm 14 is shown. It can be seen that the second gripping pad 18 is disposed near the distal end along an enlarged (i.e., wide) portion of the second arm 14. The tip portion 22 on the second arm 14 is located opposite the tip portion 20 of the first arm for providing jaws for gripping tissue or other material. The second gripping pad 18 is preferably elongated in the longitudinal direction similar to the first pad. In the illustrated embodiment, each of the resilient gripping pads is formed with a plurality of oval shaped ridges 54 with grooves 56 therebetween. The grooves 56 formed in the second gripping pad 18 preferably receive the ridges 50 formed in the first gripping pad 16 and vice versa.

With reference now to FIG. 7, a cross-sectional view along the distal end portion of the second arm 14 is provided. In one preferred embodiments the gripping pad 18 has a thickness of about 0.025 inches and the arm 14 has a thickness of about 0.050 inches. Such dimensions are merely exemplary and the gripping pads and arms of course can have other thickness as well. It can be seen that the thickness of the arm 14 may vary beneath the gripping pad 18 to provide teeth 58 for enhanced gripping strength. With reference now to FIG. 8, an enlarged side view of the distal end portions of the first and second arms is provided.

With reference now to FIG. 8A, in accordance with another embodiment, a surgical forceps may be provided with a first arm 14 and a second arm 16 similar to that of FIG. 8. However, in this embodiment, no gripping pad is provided along the first arm 14. Instead, a relatively hard gripping surface 16A is provided along the distal end portion of the first arm for engagement with the opposing gripping pad 18. The hard gripping surface 16A may be attached to the inner face of the first arm by a variety of different methods, such as welding, adhesives, plating, coating or chemical vapor deposition. Alternatively, the inner face of the first arm may itself be formed or textured to provide the gripping surface. It can be seen that the gripping surface 16A may be formed with teeth or ridges for complementing the shape of the gripping pad 18. Although the above embodiments are illustrated as having ridges and grooves along the gripping pads and/or gripping surfaces, it will be appreciated that a wide variety of other textured surfaces may be used. Alternatively, one or both may be formed with a substantially flat surface. FIG. 8A also illustrates an alternative configuration for the opposing tips 20A, 22A wherein the tips are substantially parallel and may be configured with a clearance therebetween when the forceps are in the closed position.

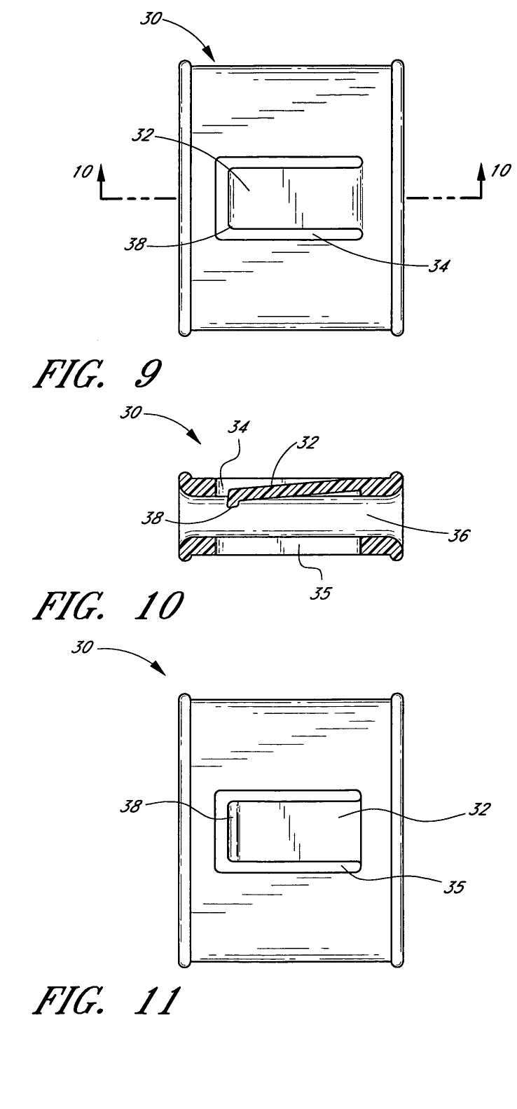

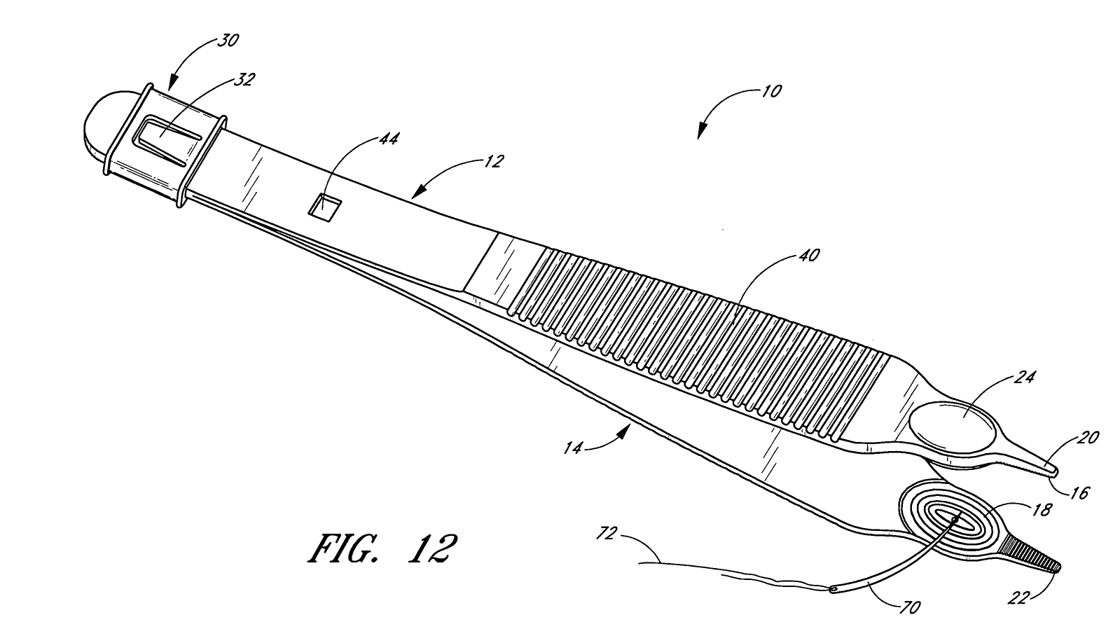

With reference to FIGS. 9 and 10, the locking mechanism 30 will now be described in greater detail. FIG. 9 is a top view illustrating the locking mechanism 30 in isolation. The retaining mechanism 32 is preferably provided along a central portion of the locking mechanism 30. A gap 34 extends along three sides of the retaining mechanism 32 such that the retaining mechanism is configured to flex up and down in a cantilever fashion. As best seen in FIG. 10, the retaining mechanism 32 preferably includes a lip portion 38 adapted to slide along the outer surface of the first arm when in the open position. To achieve this, the retaining mechanism 32 is preferably biased downward toward the arm. When the locking mechanism 30 is slid forward, the retaining mechanism 32 snaps into the hole (see element 44 in FIG. 1) formed into the first arm. Once the retaining mechanism 32 is received within the hole, the lip portion 38 engages the proximal edge of the hole for preventing the locking mechanism from sliding in a proximal direction, thereby maintaining the forceps in the closed position.

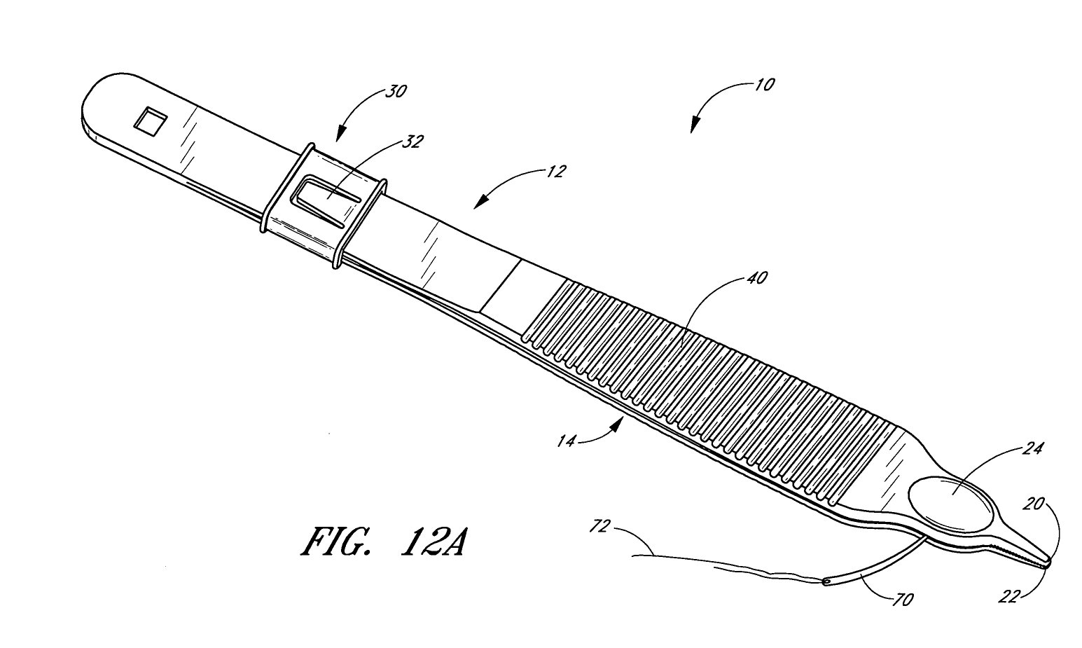

With reference to FIG. 10, it can be seen that the locking mechanism 30 is preferably formed with a central passage 36 sized for receiving the proximal end portions of the first and second arms. As described above, the retaining mechanism 32 is biased in a downward position such that it snaps into the hole in the first arm. With reference to FIG. 11, the bottom side of the locking mechanism 30 is shown. In the illustrated embodiment, the bottom side is formed with a hole 35. Although one particular embodiment of a locking mechanism is illustrated, a variety of other locking mechanisms are contemplated to fall within the scope of the present invention. For example, as will be discussed in more detail below, a clasp may be rotatably attached to one of the arms wherein the clasp may be manipulated by the clinician to engage the opposing arm when it is desired to lock the forceps in the closed position.

With reference again to FIG. 1, when it is desired to grasp an object, the clinician holds the surgical forceps 10 and applies a force on the first and second arms 12, 14, such as by squeezing with a thumb and forefinger. In various preferred applications, the clinician urges the arms together for grasping a suturing needle, tissue or another article, between the first and second arms. When the forceps is used to grasp a suturing needle, the first and second resilient gripping pads 16, 18 deform around the needle body. As a result, the gripping pads advantageously provide an even gripping force along the needle and reduce the likelihood of undesirable needle movement (e.g., rolling) while being gripped. As discussed herein, in certain preferred embodiments, complementary ridges and/or grooves are provided along the first gripping pad 16 of the first arm 12 and the second gripping pad 18 of the second arm 14 for further enhancing the gripping ability.

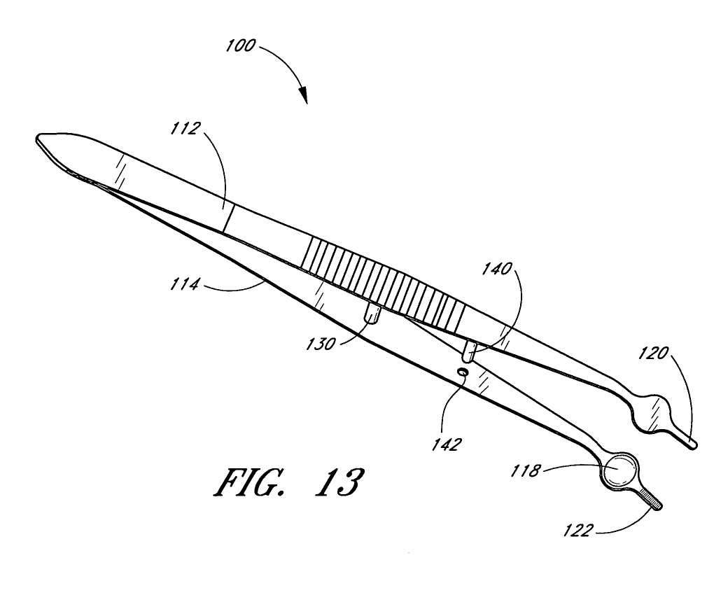

For purposes of illustration, FIG. 12 illustrates the surgical forceps 10 in combination with a suturing needle 70. The illustrated suturing needle has a curved shape configured for enabling the clinician to pull a suture thread 72 through tissue or other material. The suturing needle 70 is shown adjacent the resilient gripping pad 18 on the second arm 14. As shown, the distal tip portion of the suturing needle 70 is located within the central region of the gripping pad 18. When the first and second arms are squeezed together, the first gripping pad 16 presses the needle against the second gripping pad 18 for securely holding the needle. FIG. 12A illustrates the forceps in the closed and locked position with the distal tip of the suturing needle captured and held between the gripping pads.

The gripping pads 16, 18 are preferably formed with relatively large sizes for providing the ability to completely enclose the tip of the needle 70. Accordingly, in a particularly advantageous feature, the forceps 10 protect the clinician and patient from the distal tip of the needle 70 and thereby help prevent accidental needle sticks. Furthermore, it will be appreciated that the locking mechanism 30 provides an improved device and method for safely disposing of the suturing needle. Using the locking mechanism, the suturing needle may be permanently and safely retained within the forceps for safe disposal at the completion of the procedure. Locking is achieved by sliding the locking mechanism 30 forward (i.e., distally) until the retaining mechanism 32 snaps into the hole 44 in the first arm 12. In this position, the locking mechanism 30 will continue to hold the first and second arms 12, 14 together in the absence of any force applied by the clinician. With the locking mechanism in place, as shown in FIG. 12A, the surgical forceps 10 and the needle 70 may be disposed of as a single unit. The pointed tip of the needle is preferably fully contained within the perimeters of the gripping pads at the time of disposal to reduce the risk of a needle prick during disposal and processing of the waste.

The first and second arms 12, 14 of the forceps 10 are made from any suitable material, such as, but not limited to, molded plastic, stainless steel, tungsten carbide, carbide steel, or ceramic. The proximal ends of the first and second arms may be joined using a pin, welding, adhesives, or any other means or methods adapted for securely coupling the arms together while allowing the distal ends of the arms to move together for gripping an object. Alternatively, the first and second arms may be formed as a single unit, such that movement occurs due to flexing of the arms.

The resilient gripping pads 16, 18 are preferably made, at least in part, from a compressible (e.g., elastomeric) material that will conform about an object when a compressive or gripping force is applied to the forceps. The compressibility of the material prevents damage to the object and/or to the forceps. When the force is removed, the material preferably returns to its original shape. In preferred embodiments, the compressible material has a high coefficient of friction for securely holding the object with little or no slippage. The compressibility and high coefficient of friction provide a substantial advantage over forceps having a substantially non-compressible material (e.g., tungsten carbide) along the inner surfaces. For example, when gripping a needle with a non-circular cross-section, the resilient pads will conform around the needle, thus reducing the likelihood of the needle rolling or shifting position while being gripped.

It has been found that a number of different grades (e.g., FDA grade) of Kraton® polymers have properties that are particularly well-suited for use as a gripping pad. Other preferred materials for gripping pads include, but are not limited to, other thermoplastics elastomers, latex, silicone, and urethane epoxies. In one preferred embodiment, the gripping pads are formed of a Kraton® compound, such as, for example, Dynaflex®, which is available commercially from GLS Corporation. The Dynaflex compound used in the preferred embodiment has a Shore A hardness of about 28 durometer. Suitable materials preferably have a hardness of about 100 or less durometer, and more preferably no more than about 75 durometer. Dynaflex can be disposed into the recess of the expanded area 19 on the arms 12, 14 through traditional injection molding processes, including an over-molding process, or can be separately formed and then attached (e.g., using a suitable adhesive) to the arm.

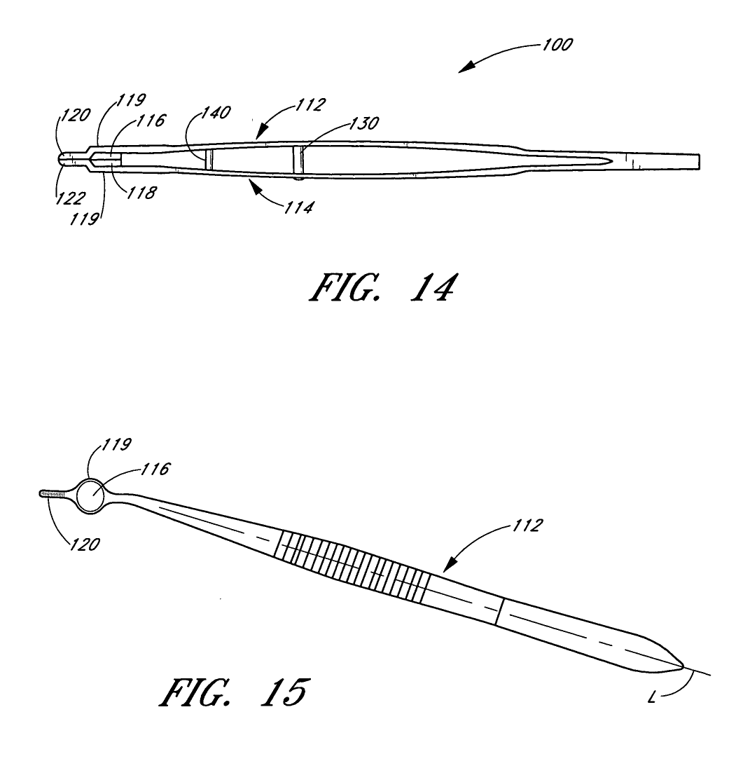

With reference now to FIG. 13, another embodiment of a surgical forceps 100 generally includes a first elongate arm 112 and a second elongate arm 114 that are joined along proximal ends thereof. The forceps 100 further comprises a clasp 130 for locking the arms 112, 114 in the closed position. With reference to FIG. 14, a side view of the forceps 100 is shown wherein the clasp 130 on the first arm 112 is engaging the second arm 114 such that the forceps is locked in the closed position. In one variation, the clasp may be formed with one or more teeth for engaging the opposing arm in a ratcheting manner. The forceps 100 also preferably includes an alignment pin 140 for ensuring proper alignment of the arms when squeezed together.

The distal end portions of the arms 112, 114 are preferably bent at an angle of approximately 15 degrees with respect to a longitudinal axis L as best shown in FIG. 15 wherein the first arm 112 is shown in isolation. The bent shape enhances the clinician's ability to access and see the treatment site during use. In alternative variations, the distal end portions of the arms can be aligned with the longitudinal axis L or can be skewed at other angles relative to the longitudinal axis. With continued reference to the illustrated embodiment of FIGS. 13 through 15, the first and second arms 112, 114 are provided with gripping surfaces along the inner surfaces comprising first and second resilient gripping pads 116, 118, respectively. The gripping pads 116, 118 are preferably located along expanded sections 119 on the first and second arms 112, 114. The expanded sections preferably have the same general shape as the gripping pads 116, 118, but are slightly larger in size. However, one or more of the gripping pads 116, 118 can be larger than and/or can have a different shape than the corresponding expanded section(s) 119 of the arms.

With particular reference now to FIG. 15, the inner face of the first arm 112 is shown. While the inner face of the second arm 114 is not illustrated, its shape and structure corresponds with and is similar to that of the first arm 112. Accordingly, the following description of the first arm 112 and the gripping pad 116 on the first arm 112 should be understood to apply equally to the structure of the second arm 114 and second gripping pad 118, unless otherwise noted. The first gripping pad 116 preferably has a generally circular or oval shape; however, other shapes are also possible. As can be seen, the width of the pad 116 is considerably larger (e.g., more than three times larger in the illustrated embodiment) than the width of the adjacent portions of the arm 112. The relatively large pad size advantageously allows an object to be gripped between the pads in a secure manner wherein the gripping force is applied firmly and evenly along a substantial length of the object. The relatively large pad size also allows the clinician to completely enclose the distal end portion of the needle between the gripping pads. When the distal needle tip is fully enclosed between the gripping pads, the possibility of an accidental needle stick during the application of suture is substantially reduced or eliminated completely.

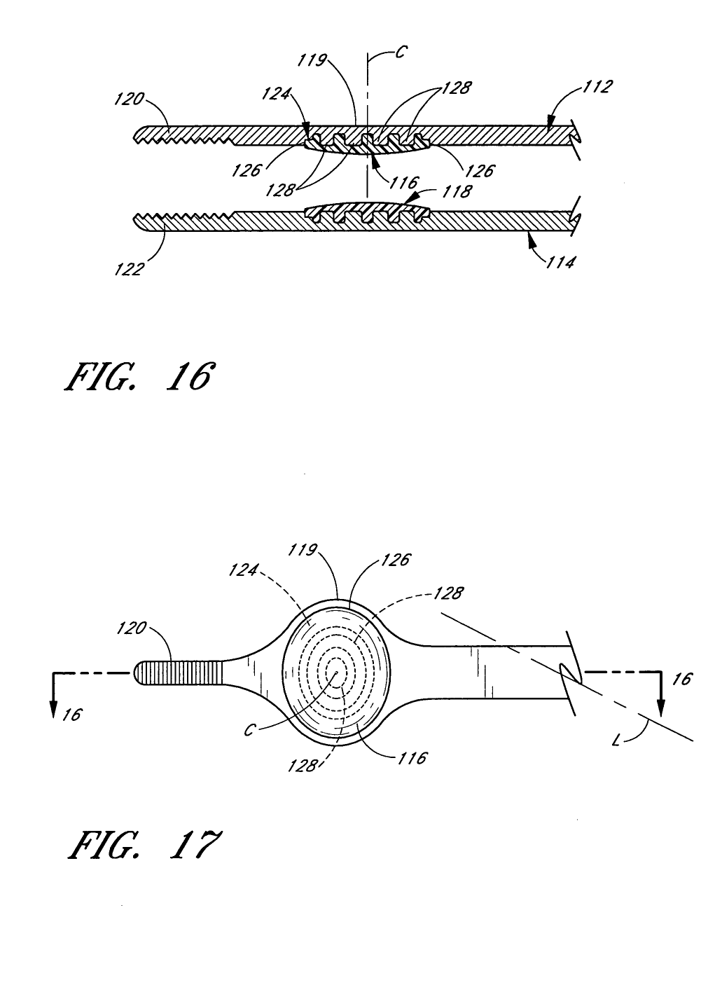

With reference now to FIG. 16, an enlarged cross-sectional view of the distal end portions of the first and second arms 112, 114 is provided. It can be seen that serrated teeth are provided along the inner faces of the distal tips 120, 122. The teeth may be formed into the material of the arm itself or the teeth may be formed of another material, such as, for example, an elastomeric material coupled to the distal tip. In another configuration, an elastomeric coating may be provided over the teeth. If teeth are provided on both distal tips, it is desirable for the teeth to be arranged in a mating configuration. In addition to teeth, it will be appreciated that a wide variety of other textures may be provided along the distal tip of one or both arms. Still further, the distal tips may be formed without any teeth or texturing.

With reference to FIGS. 16 and 17, particular advantageous features of the first gripping pad 116 will be described with the understanding that the description of the first arm 112 and the first gripping pad 116 applies equally well to the structure of the second arm 114 and the second gripping pad 118. It can be seen that the first gripping pad 116 is disposed generally within a depression or recess 124 defined along the expanded section 119 on the arm 112. The recess 124 includes a wall 126 that lies generally normal to the longitudinal axis. In the illustrated embodiment, the wall 126 has a generally annular or oval shape about a central axis C, which lies generally normal to the longitudinal axis. The wall 126 thus forms a rigid outer rim that surrounds a perimeter edge of the resilient gripping pad 116. As best seen in FIG. 16, the gripping pad 116 has a substantial thickness such that the surface of the pad is raised above the inner surface of the first arm 112. Accordingly, the gripping pad, rather than the surrounding expanded section 119 of the arm 112, contacts the article grasped with the forceps.

The wall 126 also advantageously inhibits expansion of the resilient material in a radial direction relative to the central axis C of the pad 116. In other words, the wall 126 will inhibit the resilient material from spreading radially over the inner surface of the arm 112. As a result of the wall 126, when a gripping (i.e., compressive) force is applied to the surface of the pad 116, the resilient material will deform substantially outwardly from the recess 124 and around the object toward the opposing pad. Consequently, the resilient material will tend to conform to the shape of the retained article (e.g., a suturing needle), thereby increasing the surface contact between the resilient material and the article held by the surgical forceps 100.

Rigid intrusions 128 may be provided along the inner surface of the recess in the arm 112 for protruding into the resilient material. The intrusions 128 can be integrally formed with the arm 112 or can be disposed within the recess before the pad 116 is attached (e.g., by an adhesive) or formed (e.g., by an over-molding process). In the illustrated embodiment, the intrusions take the form of annular ridges. However, it will be appreciated that the intrusions may take a variety of other forms as well. As best illustrated in FIG. 16, the tops of the intrusions 128 lie beneath the surface of the gripping pad 116. The intrusions 128 are shaped for creating variable compressibility within the resilient gripping pad. Accordingly, the resilient material is allowed to deform about a portion of the article for enhanced gripping while the intrusions provide a rigid substrate for engaging and pressing against the article to securely hold the article within the forceps.

In the illustrated embodiment, the exposed side of each pad preferably has a substantially smooth outer surface. However, in alternative configurations, one or both of the pads 116, 118 may have a textured surface. For example, a series of concentric ribs can be provided along the surface of the one or both of the pads 116, 118 for enhanced gripping ability. Additionally, in certain variations, each of the pads may be formed with a different surface texture.

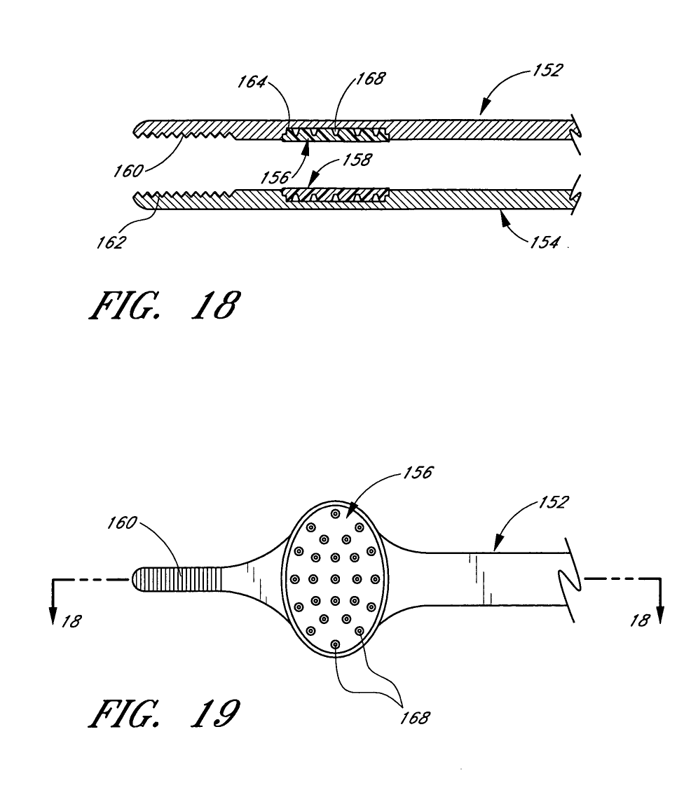

With reference now to FIG. 18, an enlarged cross-sectional view of alternative gripping pads 156, 158 are shown disposed along first and second arms 152, 154 of a surgical forceps. First and second opposing tips 160, 162 are provided along the distal end portions of the first and second arms. With reference to FIGS. 18 and 19, the gripping pad 156 along the distal end portion of the first arm 152 will be described in more detail. However, the description of the first arm 152 and the first gripping pad 156 may apply equally well to the structure of the second arm 154 and the second gripping pad 158. In this embodiment, intrusions into the resilient material are provided as a plurality of frusto-conically shaped members 168 disposed over the inner base surface of a recess 164 formed in the first arm. The frusto-conically shaped members 168 are preferably substantially rigid in construction. A layer of deformable material of the first pad 156 is provided over and/or around the members 168. It has been found that the resulting composite structure is particularly advantageous for gripping and retaining a suturing needle. In particular, the deformable material of the first gripping pad 156 conforms around the shape of the needle (or other object) and the members 168 provide a way to firmly grip the object and to force the gripping pad 156 at least partially around a portion of the needle. Still further, the location and arrangement of the members provide the deformable material 156 with particularly desirable compressibility characteristics. In particular, the members 168 provide the gripping pad 156 with a gradually increasing stiffness that is desirable during use. FIG. 19 illustrates the members 168 arranged in one preferred configuration wherein the members 168 can be seen beneath the gripping pad. In other preferred embodiments, the deformable material of the gripping pad 156 illustrated in FIGS. 18 and 19 may face a textured surface provided along the arm. Still further, it will be appreciated that alternative forceps embodiments may be constructed without intrusions.

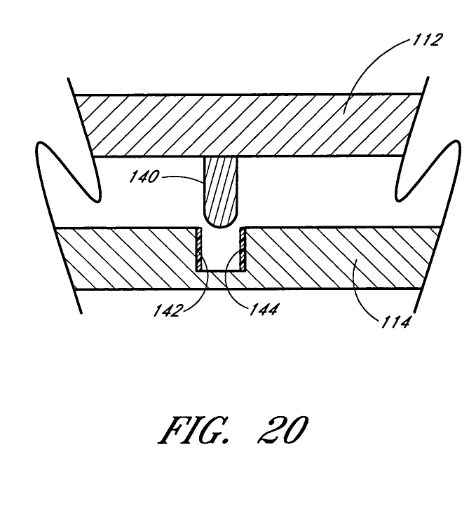

With reference again to the surgical forceps 100 of FIG. 13, the alignment pin 140 is provided for ensuring proper alignment between the first and second arms 112, 114. More particularly, the alignment pin 140 prevents the arms from slipping laterally relative to one another when squeezed together. With reference now to FIG. 20, an enlarged view of the alignment pin 140 is provided wherein the pin is disposed along the first arm 112 and a corresponding recess 142 disposed along the second arm. The recess 142 is sized for receiving the pin 140 when the arms are squeezed together. A bushing 144 is preferably provided along the interior of the recess 142. The bushing advantageously reduces frictional resistance. The bushing may be helpful because frictional resistance may result in an undesirable loss of tactile feel to the clinician during use.

With reference again to FIG. 13, the clasp 130 provides a locking mechanism for maintaining the first and second arms 112, 114 in the closed position. As best illustrated in FIG. 14, the clasp 130 is disposed along the first arm 112 for engagement with the second arm 114. When the arms are squeezed together, the clasp 130 on the first arm 112 is configured to engage and hold the second arm 114 for use as a locking mechanism. The clasp 130 is preferably attached to the first arm 112 in a rotatable or bendable manner for allowing movement of the clasp relative to the first arm. A receiving portion, such as an indentation or other mechanism, may be provided on the second arm 114 for receiving and holding the clasp 130.

During use, the clinician applies a force to grasp and retain an article, such as a suturing needle. If desired, the clinician may increase the force applied to the arms 112, 114 such that the middle portions of the arms deform inward and thereby come together. When a sufficient force is applied, the clasp 130 will contact the second arm 114, thereby causing the clasp to bend outward and advance over the second arm 114. After sufficient advancement, the clasp 130 will slide into the receiving portion 132, as shown in FIG. 14 for locking the forceps 100 in a closed position. The clasp 130 is preferably biased and shaped such that the clasp 130 will snap over the second arm into the locked position. The clasp may include a number of teeth or other engaging members for allowing the arms to be locked together in a variety of different positions.

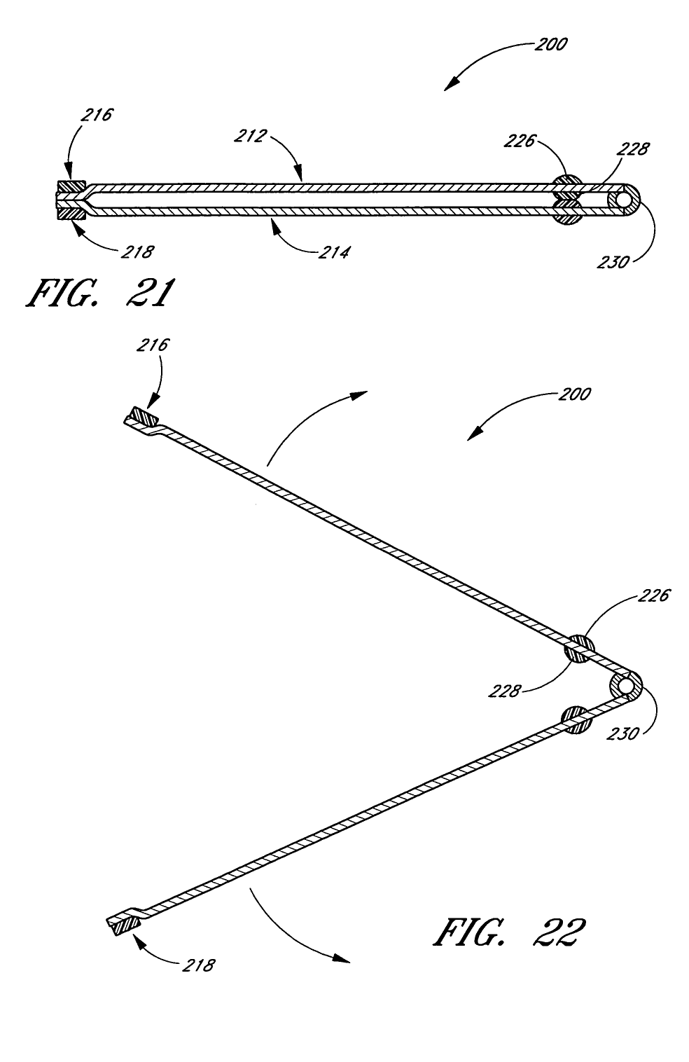

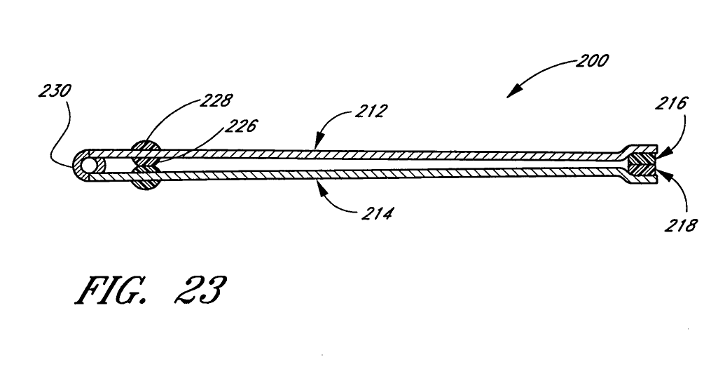

FIG. 21 illustrates yet another preferred embodiment of a surgical forceps 200 wherein first and second arms 212, 214 are rotatably coupled along the proximal ends. As shown in FIG. 22, the arms 212, 214 are preferably rotatably coupled with a biased hinge 230 such that the arms may be folded back on themselves. This embodiment allows the clinician to quickly and easily reverse the inner and outer faces of the arms of the forceps to a second configuration, as shown in FIG. 23. In this embodiment, each arm may have a first face provided with teeth (not shown) adapted for gripping tissue and a second face provided with gripping pads 216, 218 of the type discussed above. The clinician can thereby easily reconfigure the forceps according to the desired use. In a first configuration, the forceps may be used to grip an object between the hard surfaces along the first faces. In a second configuration, the forceps may be used to grip the object between the resilient gripping pads 216, 218 disposed along the second faces. Rather than hard surfaces and gripping pads, a variety of other gripping surfaces may also be used with this forceps embodiment without departing from the scope of the invention.

In one preferred configuration, deformable members 226, 228 may be provided along the first and second faces of each arm 212, 214 instead of, or in addition to, the spring biased hinge 230. Each deformable member is adapted to contact an opposing deformable member for providing a fulcrum that urges the arms apart in the absence of a gripping force. However, the deformable members are sufficiently malleable such that the outward biasing force provided by the deformable members may be overcome by the clinician during use. The deformable members are preferably made of an elastomeric material.

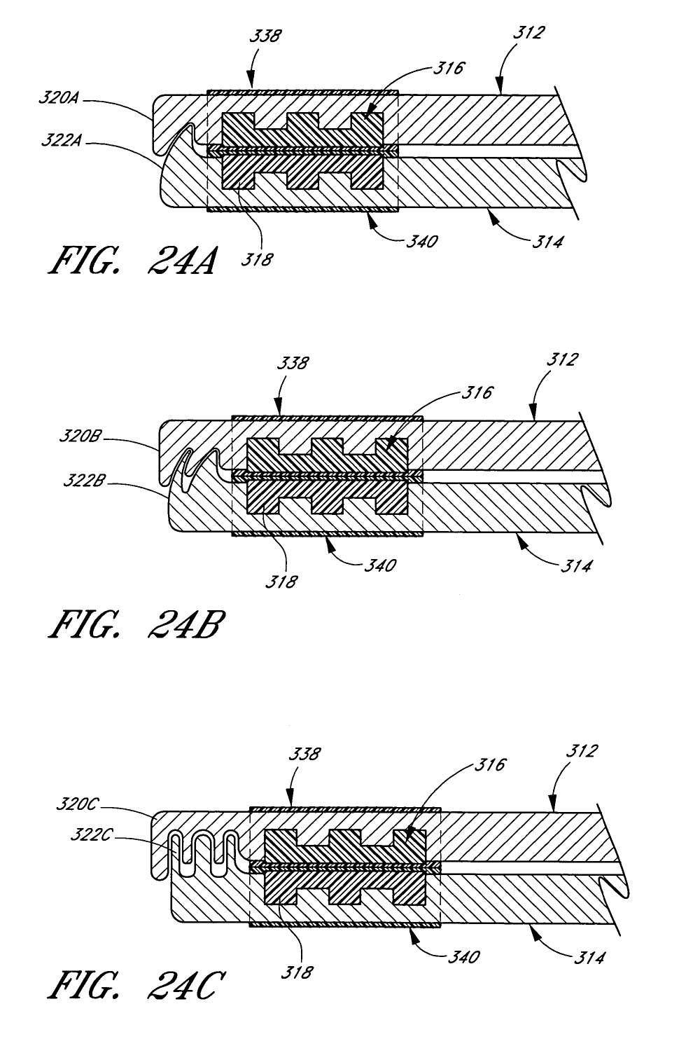

In still other embodiments, a variety of tip configurations may be used with a surgical forceps constructed according to the present invention. With reference to FIG. 24A, a side cross-sectional view of the distal end portion of a forceps is shown for illustrating another preferred method of attaching a gripping pad to an arm. In the illustration configuration, the first gripping pad 316 is provided as part of a sleeve 338 that extends over the distal end portion of the first arm 312. Similarly, the second gripping pad 318 is provided as part of a sleeve 340 that extends over the distal portion of the second arm 314. The sleeves are preferably tightly fit such that the sleeves are fixed with respect to the arms during use. In addition or in the alternative, the sleeves 340 can interact with the structure of the arm themselves (e.g., fit within reliefs on the arm) to inhibit slipping of the sleeves relative to the arms.

FIG. 24A also shows a first alternative tip embodiment wherein the opposing tips 320A, 322A are each formed with a single tooth. FIG. 24B illustrates a second alternative tip embodiment wherein the opposing tips 320B, 322B are each formed with two teeth. FIG. 24C illustrates a third alternative tip embodiment wherein the opposing tips 320C, 322C are each formed with three teeth. In these and other similar embodiments, the teeth cooperate to provide enhanced gripping. The opposing teeth preferably overlap when the arms are squeezed together.

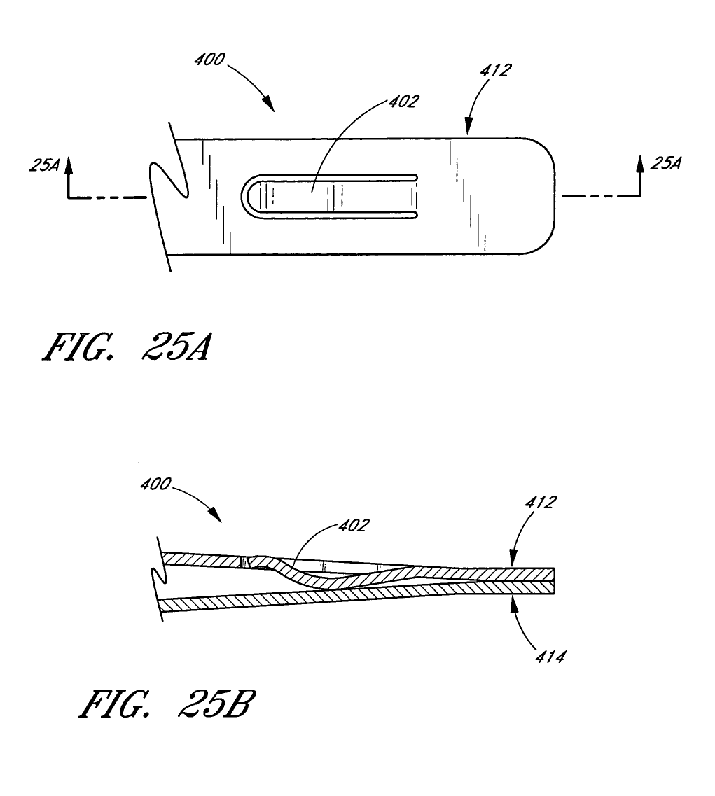

With reference now to FIGS. 25A and 25B, a preferred biasing mechanism 400 is illustrated for maintaining the arms 412, 414 in an open position in the absence of a gripping force. With reference to the top view of FIG. 25A, the biasing mechanism 400 comprises a curved member 402 that is formed into the first arm 412. As best seen in the cross-sectional view of FIG. 25B, the curved member 402 is preferably located at the proximal end of the first arm 412 and bows inward for engagement with the proximal end of the opposing arm. As a result, the curved member 412 provides a biasing force that urges the arms apart in the absence of an external force. The curved member 412 is configured to deform such that the biasing force can be overcome by the clinician during use.

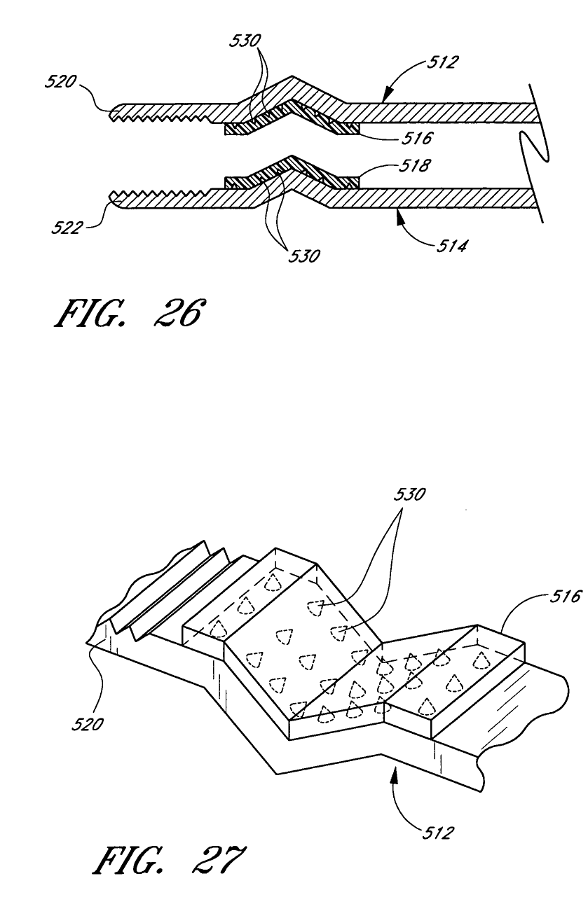

With reference now to FIGS. 26 and 27, the distal end portion of yet another preferred embodiment of a surgical forceps is illustrated. With reference to FIG. 26, a cross-sectional view illustrates first and second arms 512, 514 formed with first and second contoured gripping pads 516, 518. Each arm is preferably formed with complementary angular regions adjacent to the first and second gripping pads 516, 518. First and second opposing tips 520, 522 are provided distal to the gripping pads. In this embodiment, the first gripping pad 516 is formed with a V-shape to provide a notch well suited for maintaining a suturing needle in a fixed location. The second gripping pad 518 has a complementary shape for mating with the first pad. In addition, the first and second arms 512, 514 may be provided with a plurality of teeth 530, 532 disposed beneath the first and second gripping pads 516, 518. The gripping pads 516, 518 may comprise an elastomeric material disposed over the teeth 530, 532. The teeth are provided for preventing the needle from slipping out of the notch and also help urge the elastomeric material to deform around the object held within the forceps.

Although this invention has been disclosed in the context of certain preferred embodiments and examples, it will be understood by those skilled in the art that the present invention extends beyond the specifically disclosed embodiments to other embodiments and/or uses of the invention and obvious modifications and equivalents thereof. In particular, while the present surgical forceps has been described in the context of particularly preferred embodiments, the skilled artisan will appreciate, in view of the present disclosure, that certain advantages, features and aspects of the forceps may be realized in a variety of other applications, many of which have been noted above. Additionally, it is contemplated that various aspects and features of the invention described can be practiced separately, combined together, or substituted for one another, and that a variety of combination and sub-combinations of the features and aspects of the described embodiments can be made and still fall within the scope of the invention. Thus, it is intended that the scope of the present invention herein disclosed should not be limited by the particular disclosed embodiments described above.

1. A forceps for gripping and retaining a surgical needle, comprising:
first and second arms having proximal and distal end portions, the arms being joined together at least near the proximal end portions; and
first and second gripping surfaces disposed along the distal end portions of the first and second arms, respectively, the first gripping surface comprising a first resilient gripping pad;
wherein the gripping surfaces are disposed in an opposing relationship for gripping an object therebetween when a gripping force is applied to the forceps.

2. The forceps of claim 1, wherein the first resilient gripping pad is provided with a textured surface.

3. The forceps of claim 2, wherein the textured surface comprises a plurality of concentric oval ridges.

4. The forceps of claim 1, wherein a plurality of intrusions is disposed beneath the first resilient gripping pad.

5. The forceps of claim 1, wherein the second gripping surface comprises a second resilient gripping pad.

6. The forceps of claim 1, further comprising a locking mechanism for selectively maintaining the gripping surfaces in close proximity when the gripping force is removed.

7. The forceps of claim 6, wherein the locking mechanism comprises a ring-shaped member disposed along the proximal end portions of the first and second arms, the ring-shaped member being configured for longitudinal movement.

8. The forceps of claim 7, wherein the ring-shaped member further comprises a retaining mechanism and the first arm is formed with an opening configured for receiving the retaining mechanism for holding the ring-shaped member in the locked position.

9. The forceps of claim 6, wherein the locking mechanism comprises a clasp coupled to the first arm and configured to engage a portion of the second arm.

10. The forceps of claim 6, further comprising an alignment pin disposed along the inner surface of the first arm and a corresponding hole in the second arm, wherein the alignment pin is receivable in the hole when the forceps are in a closed position to maintain the first and second arms in a proper alignment.

11. The forceps of claim 1, wherein the first and second arms further comprise first and second opposing tips located distal to the first and second gripping pads.

12. The forceps of claim 11, wherein the first and second gripping surfaces are substantially wider than the first and second opposing tips.

13. The forceps of claim 1, wherein the first resilient gripping pad conforms to the shape of the object being held.

14. The forceps of claim 13, wherein the first gripping pad is formed of a Kraton® compound.

15. The forceps of claim 1, wherein the first and second arms are joined at the proximal end portions.

16. The forceps of claim 1, wherein the first and second arms are rotatably coupled.

17. The forceps of claim 1, wherein each of the first and second arms includes an expanded area along the distal end portion on which the corresponding gripping surface is disposed.

18. The forceps of claim 1, further comprising a biasing mechanism that biases the distal end portions of the first and second arms apart in the absence of the gripping force.

19. A forceps for gripping and retaining a surgical needle, comprising:
first and second arms having proximal and distal end portions, the arms being joined together at least near the proximal end portions;
first and second gripping surfaces disposed on the first and second arms in an opposing relationship for holding an object therebetween when a gripping force is applied to the forceps; and
a locking mechanism for selectively maintaining the distal end portions of the first and second arms in close proximity for gripping the object when the gripping force is removed.

20. The forceps of claim 19, wherein the locking mechanism comprises a clasp.

21. The forceps of claim 19, wherein the locking mechanism comprises a slidable ring disposed around the proximal end portions of the first and second arms.

22. The forceps of claim 19, wherein the first gripping surface comprises a first gripping pad and the second gripping surface comprises a second gripping pad, at least one of the gripping pads having a resilient material that conforms to the shape of the object being held.

23. The forceps of claim 22, wherein the resilient material is provided with a textured surface.

24. The forceps of claim 22, wherein each of the firsts and second arms includes an expanded area along the distal end portion on which the corresponding gripping pad is disposed.

25. The forceps of claim 19, further comprising a biasing mechanism that biases the distal end portions of the first and second arms apart in the absence of the gripping force.

26. The forceps of claim 22, wherein the first gripping pad is softer than the second gripping pad.

27. A forceps for gripping and retaining a surgical needle, comprising:
first and second arms having proximal and distal end portions, the arms being joined together at least near the proximal end portions; and
first and second gripping surfaces disposed at first and second enlarged regions located along the distal end portions of the first and second arms, respectively;
wherein the gripping surfaces are disposed in an opposing relationship for gripping an object therebetween when a gripping force is applied to the forceps.

28. A surgical forceps, comprising:
a first arm having a proximal end and a distal end, the first arm having first and second faces disposed on opposite sides of the distal end;
a second arm having a proximal end and a distal end, the second arm having first and second faces disposed on opposite sides of the distal end;
wherein the proximal ends of the first and second arms are rotatably coupled such that the forceps has a first configuration for gripping an object between the first faces of the first and second arms and the forceps has a second configuration for gripping the object between the second faces of the first and second arms.

29. The surgical forceps of claim 28, wherein resilient gripping pads are disposed along the first faces of the first and second arms.

30. A method of closing a wound, comprising:
providing a surgical forceps having first and second elongate arms, the arms having first and second resilient pads disposed along inner faces in an opposing relationship;
pushing a suturing needle through a patient's tissue on opposite sides of the wound such that a distal tip of the suturing needle emerges from the tissue, the suturing needle pulling suture through the tissue;
gripping the distal tip of the suturing needle between the first and second resilient gripping pads and pulling the needle out from the tissue;
locking the surgical forceps in a closed position such that at least the distal tip of the needle is fully enclosed and protected between the resilient pads; and
disposing of the needle and forceps.
